(19) United States
(12) Patent Application Publication
(10) Pub. No.: US 2009/0240067 A1
(43) Pub. Date: Sep. 24, 2009

(54) METHOD OF PRODUCING A BISBENZODITHIOL COMPOUND

(75) Inventors: Masuji Motoki, Minami-ashigara-shi (JP); Toshihiko Yawata, Minami-ashigara-shi (JP); Naoyuki Hanaki, Minami-ashigara-shi (JP); Tomoaki Nakamura, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/408,114

(22) Filed: Mar. 20, 2009

(30) Foreign Application Priority Data
Mar. 21, 2008 (JP) 2008-074640
Jul. 18, 2008 (JP) 2008-187010

(51) Int. Cl. C07D 339/06

(52) U.S. Cl. 549/31

(57) ABSTRACT

A method of producing a compound represented by formula (1), including: allowing 1,4-benzoquinone or 1,2-benzoquinone to react with a dithiocarbamate compound represented by formula (2) in a polar solvent:

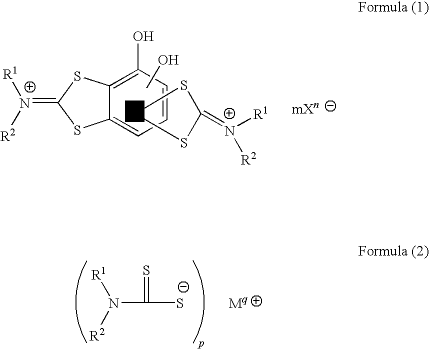

wherein R¹ and R² each independently represent a hydrogen atom, an alkyl group, an aryl group or a heterocyclic group, R¹ and R² may be the same or different and may be combined with each other to form a ring, X represents an ion necessary to neutralize the charge of the molecule, m represents an integer of 1 to 2, n represents an integer of 1 to 2, M represents a hydrogen atom, a metal atom or a conjugate acid of a base, p represents an integer of 1 to 4, and q represents an integer of 1 to 4.

### FIELD OF THE INVENTION

The present invention relates to a method of producing a bisbenzodithiol compound.

### BACKGROUND OF THE INVENTION

There is developed a compound in which two dithiol rings are condensed at the 2,3-position and 5,6-position of hydroquinone and two cyano groups are substituted at the 2-position of each of the dithiol rings via an exo-methylene group. Further, there is developed a compound in which two dithiol rings are condensed at the 2,3-position and 5,6-position of hydroquinone and a dimethylimino group is substituted at the 2-position of each of the dithiol rings. These compounds are useful for organic electronic materials, such as organic semi-conductors, for synthetic intermediates, such as ultraviolet absorbents (see, e.g., JP-A-63-150273 (“JP-A” means unexamined published Japanese patent application) and JP-A-63-225382).

It is known that the former compound described in the above can be synthesized by a method in which chloranil is allowed to react with a disodium salt that is obtained by allowing carbon disulfide to react with malononitrile in the presence of sodium hydroxide (see, e.g., Liebibs Ann. Chem., Vol. 726 (1969), pp. 103-109). Further, it is known that the latter compound described in the above can be synthesized by a method in which chloranil is allowed to react with a dimethyl ammonium salt of dimethyl dithiocarbamate that is obtained from carbon disulfide and dimethylamine (see, e.g., Tetrahedron Letters, Vol. 26 (1977), p. 2225, and ibid., Vol. 32 (1991), pp. 4897 to 4900).

Each of these synthetic methods has a problem that environmentally harmful compound, chloranil, is used as a raw material. Thus, it has been desired to develop a synthetic method in which synthesis is readily performed in a good yield using a safer raw material.

### SUMMARY OF THE INVENTION

The present invention resides in a method of producing a compound represented by formula (1), which comprises allowing 1,4-benzoquinone or 1,2-benzoquinone to react with a dithiocarbamate compound represented by formula (2) in a polar solvent:

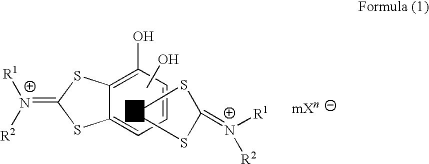

wherein R¹ and R² each independently represent a hydrogen atom, an alkyl group, an aryl group or a heterocyclic group; R¹ and R² may be the same or different and may be combined with each other to form a ring; X represents an ion necessary to neutralize the charge of the molecule; m represents an integer of 1 to 2; and n represents an integer of 1 to 2;

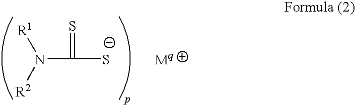

wherein R¹ and R² have the same meanings as those in formula (1); M represents a hydrogen atom, a metal atom or a conjugate acid of a base; p represents an integer of 1 to 4; and q represents an integer of 1 to 4.

Further, the present invention resides in a method of producing a compound represented by formula (3), which comprises allowing a compound represented by formula (1) to react with a compound represented by formula (4), and allowing the resultant reaction mixture to react with a compound represented by formula (5):

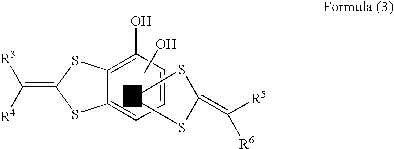

wherein R³, R⁴, R⁵ and R⁶ each independently represent a hydrogen atom or a monovalent substituent; and at least one substituent of R³, R⁴, R⁵ and R⁶ represents a substituent having Hammett's substituent constant σₚ value of 0.2 or more;

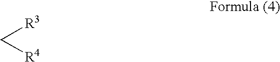

wherein R³ and R⁴ have the same meanings as those in formula (3);

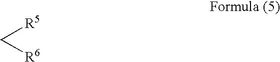

wherein R⁵ and R⁶ have the same meanings as those in formula (3).

### DETAILED DESCRIPTION OF THE INVENTION

According to the present invention, there is provided the following means:

(1) A method of producing a compound represented by formula (1), comprising: allowing 1,4-benzoquinone or 1,2-benzoquinone to react with a dithiocarbamate compound represented by formula (2) in a polar solvent:

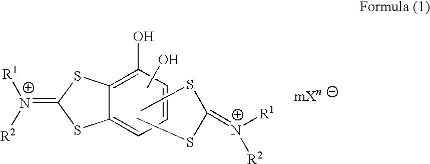

wherein R¹ and R² each independently represent a hydrogen atom, an alkyl group, an aryl group or a heterocyclic group; R¹ and R² may be the same or different and may be combined with each other to form a ring; X represents an ion necessary to neutralize the charge of the molecule; m represents an integer of 1 to 2; and n represents an integer of 1 to 2;

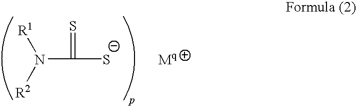

wherein R¹ and R² have the same meanings as those in formula (1); M represents a hydrogen atom, a metal atom or a conjugate acid of a base; p represents an integer of 1 to 4; and q represents an integer of 1 to 4.

(2) A method of producing a compound represented by formula (3), comprising: allowing a compound represented by formula (1) to react with a compound represented by formula (4), and allowing the resultant reaction mixture to react with a compound represented by formula (5):

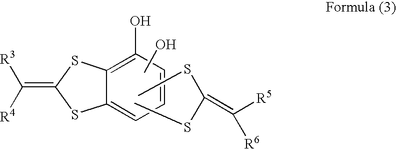

wherein R³, R⁴, R⁵ and R⁶ each independently represent a hydrogen atom or a monovalent substituent; and at least one substituent of R³, R⁴, R⁵ and R⁶ represents a substituent having Hammett's substituent constant σₚ value of 0.2 or more;

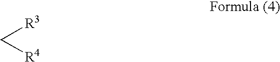

wherein R³ and R⁴ have the same meanings as those in formula (3);

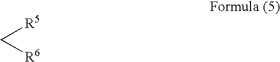

wherein R⁵ and R⁶ have the same meanings as those in formula (3).

The present invention will be explained in detail hereinafter.

The inventors have tried to perform the reaction under the similar reaction conditions as the conventional method, except for substituting 1,4-benzoquinone for chloranil that is a compound showing an intensive mutagenicity and being environmentally harmful. In the result, a compound in which a dithiol ring is condensed only at one side of hydroquinone was obtained. This is because, owing to a low solubility of the product (the compound in which a dithiol ring has been condensed only at one side of hydroquinone), crystals of the compound deposit concurrently with the formation of the target compound and hinders further reaction completely.

As a result of further intensive studies, the inventors have found that, when 1,4-benzoquinone or 1,2-benzoquinone are used as a raw material, a desired compound in which two dithiol rings are condensed at both sides of hydroquinone or catechol can be produced by preventing crystals of the synthetic intermediate from deposition by utilizing a particular solvent system for the reaction. The present invention has been attained on the basis of these findings.

The present invention relates to a method of producing a compound represented by the aforementioned formula (1). First, the compound represented by formula (1), which is the target compound of the present invention, is explained below.

In formula (1), examples of R¹ and R² include a hydrogen atom, a linear or branched alkyl group having 1 to 20 carbon atoms (preferably 1 to 10 carbon atoms, for example, methyl, ethyl, propyl, isopropyl, butyl and isobutyl), an aryl group having 6 to 20 carbon atoms (preferably 6 to 10 carbon atoms, for example, phenyl and naphthyl), a 4- to 7-membered isocyclic or heterocyclic group (preferably 5- to 6-membered ring, for example, cyclohexyl, pyridyl and morpholino), or the like. These groups may be further substituted and may be the same or different when plural substituents are present. These groups may be combined with each other to form a ring. The ring to be formed may be a saturated, or unsaturated, hydrocarbon ring, or hetero ring.

R¹ and R² are each more preferably a methyl group, an ethyl group, a propyl group, an isopropyl group, or a butyl group, and most preferably a methyl group or an ethyl group.

X represents a monoanion of hydroquinone, a dianion of hydroquinone, a mono anion of catechol, a dianion of catechol, or a conjugate base of a protic acid. X is preferably a monoanion of hydroquinone, a monoanion of catechol, an acetic acid anion, and a halogen atom, and more preferably a monoanion of hydroquinone, and a monoanion of catechol.

m represents an integer of 1 to 2 and n represents an integer of 1 to 2. Namely, the compound represented by formula (1) has m pieces of n-valence ion X with the proviso that m×n=2.

The compound represented by formula (1) includes compounds represented by formulae (1a) and (1b).

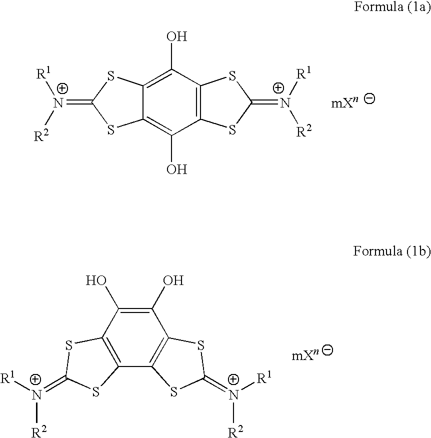

In formulae (1a) and (1b), R¹, R², X, m and n have the same meanings as those in formula (1), respectively, and the preferable ranges thereof are also the same.

Specific examples of the compound represented by formula (1) are shown below, but the present invention is not limited thereto.

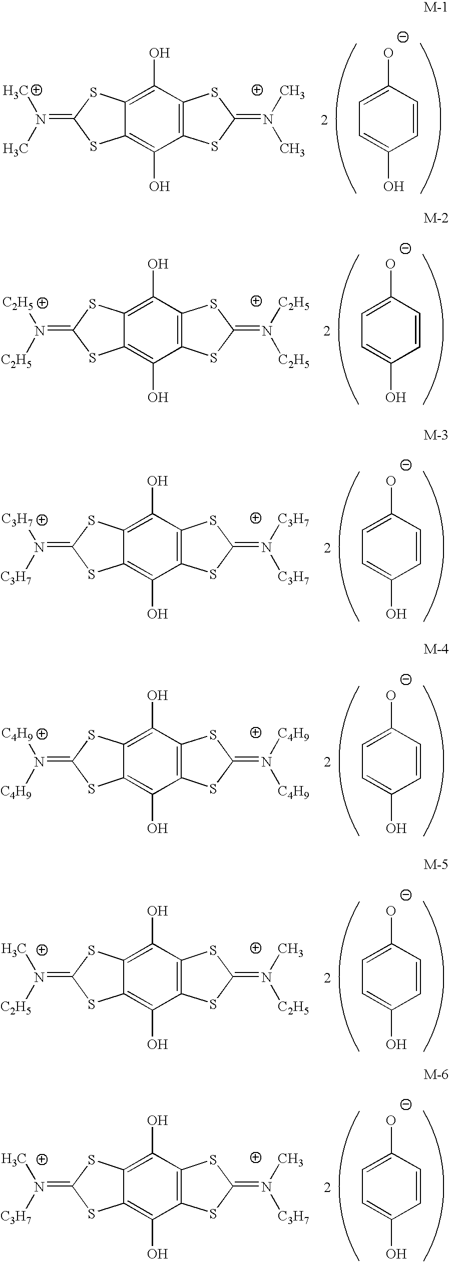

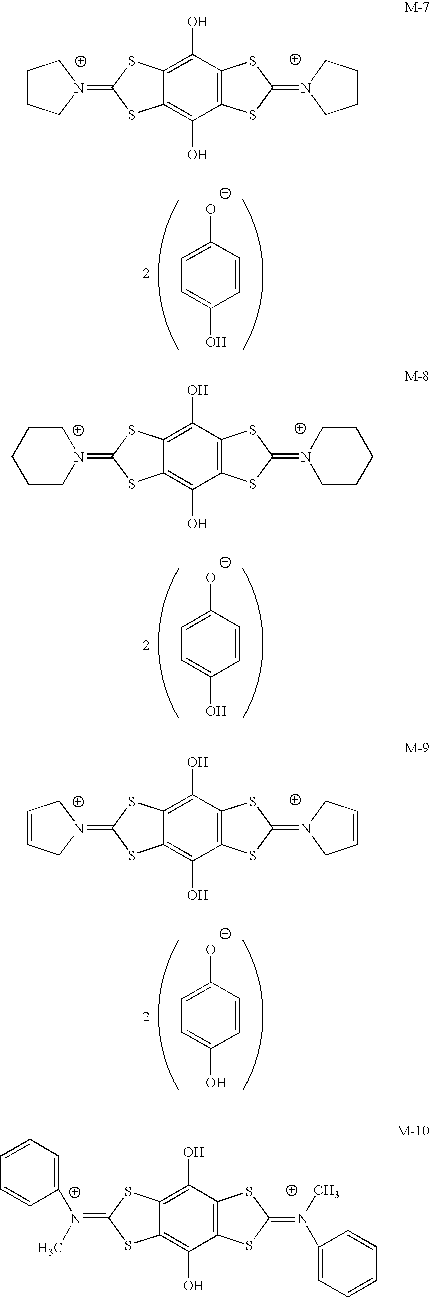

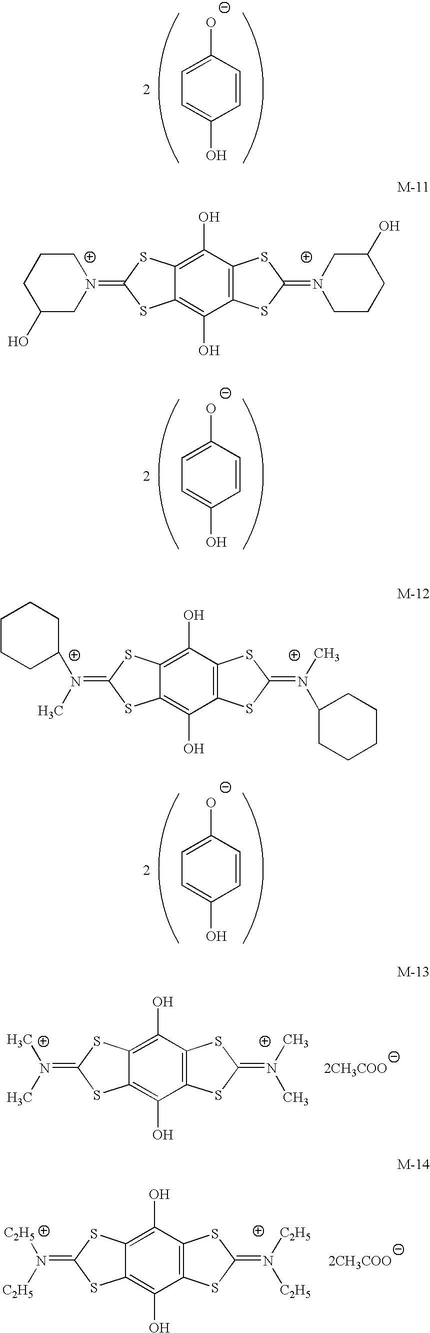

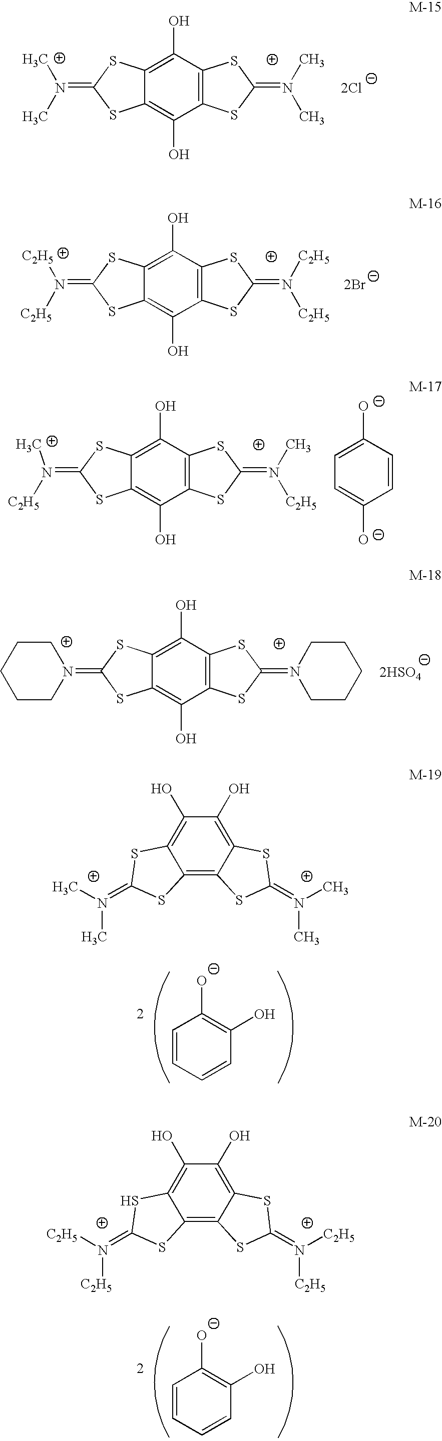

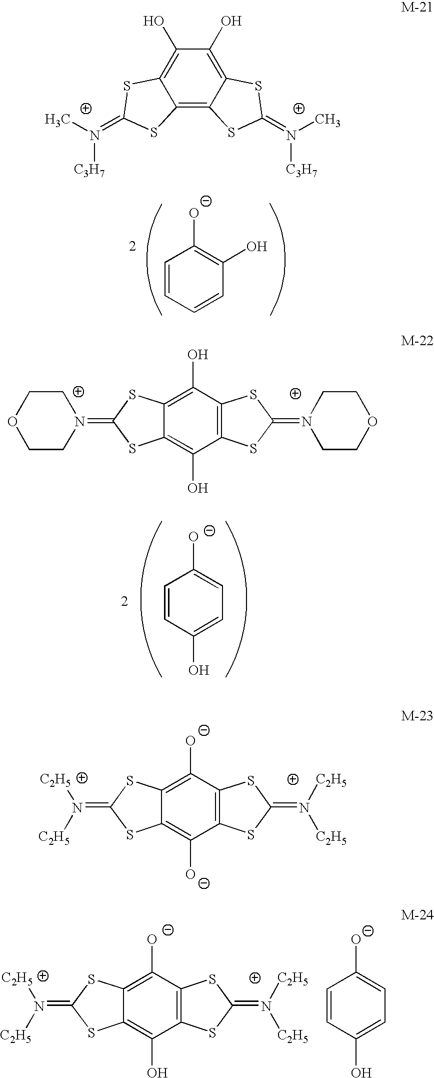

The compound represented by formula (1) may form a tautomer in accordance with the structure and the surroundings of the compound. Even though the compound is described by one of representative forms in the present specification, a tautomer that is different from the specification is also embraced in the compound represented by formula (1).

The compound represented by formula (1) may be a cationic or anionic one accompanying a proper counter ion in accordance with the structure and the surroundings of the compound. Even though the compound is described with a hydrogen ion as a counter cation, or a hydroxide ion as a counter anion in the present specification, the corresponding compound with a counter ion other than these ones is also embraced in the compound represented by formula (1). The counter ion may be a single ion, or a mixture of two or more ions having an arbitrary ratio.

The compound represented by formula (1) may have an isotopic element (such as ²H, ³H, ¹³C, ¹⁵N, ¹⁷O, or ¹⁸O).

Next, the compound represented by formula (2), which is a raw material for the compound represented by formula (1), is explained below.

In formula (2), R¹ and R² have the same meanings as those in formula (1), respectively, and the preferable ranges thereof are also the same.

M represents a hydrogen atom, a metal atom or a conjugate acid of a base. Examples of the preferable metal atom include K, Na, Li, Be, Ca, Mg, Al, Mn, Fe, Ni, Cu, B, Zn and Te. Among these, K, Na, Ca and Al are more preferable, and K and Na are most preferable. Examples of the conjugate acid of a base include ammonium, dimethyl ammonium, diethyl ammonium, pyrrolidinium, piperidinium, and pyridinium. p represents an integer of 1 to 4, and q represents an integer of 1 to 4 with the proviso that p=q.

Specific examples of the compound represented by formula (2) are shown below, but the present invention is not limited thereto.

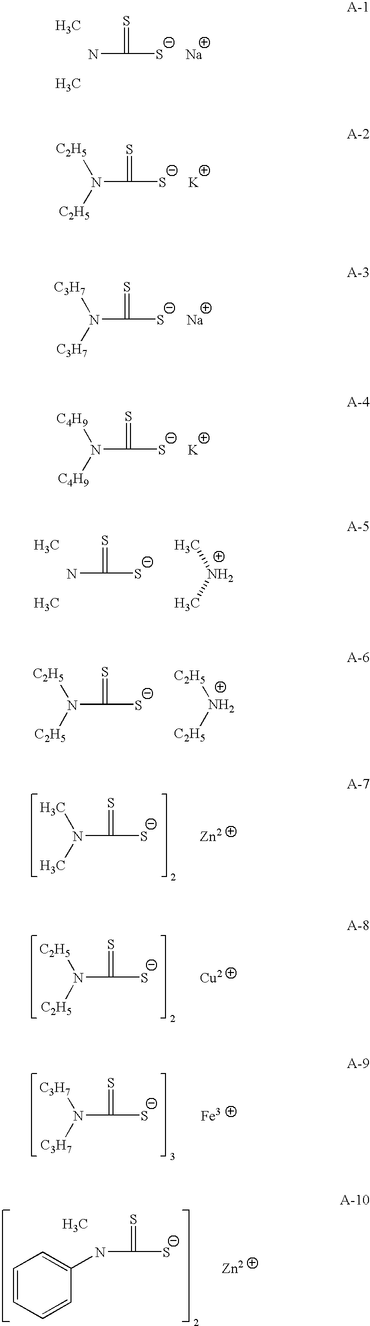

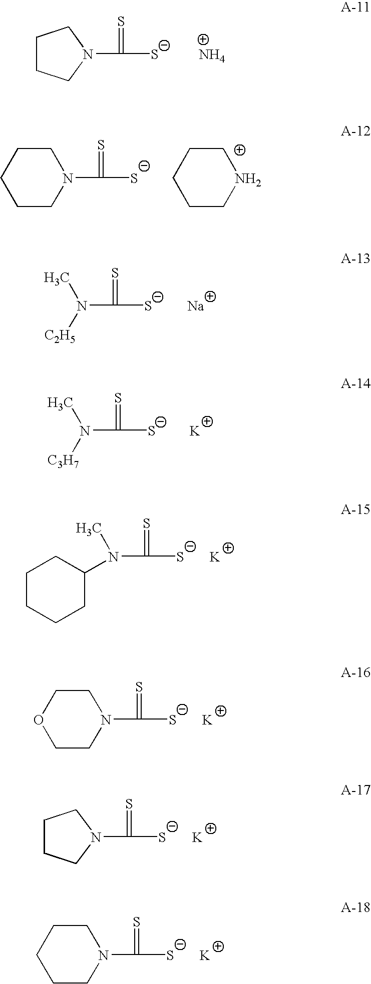

The compound represented by formula (2) can be synthesized according to any known method. The compound can be obtained by using methods described in, for example, Synthesis, Vol. 10 (1996), pp. 1193-1195, Experimentation section beginning from page 1194, left column, line 4; Journal of the Chemical Society Dalton Transactions, Vol. 9 (1992), pp. 1477-1484, Experimentation section beginning from page 1483, left column, line 33; and ibid., Vol. 4 (2000), pp. 605-610, Experimentation section beginning from page 606, left column, line 15.

For example, Exemplified compound (A-1) can be synthesized by adding both carbon disulfide and an aqueous solution of sodium hydroxide to a methanol solution of N,N-dimethylamine hydrochloride to react them. Exemplified compound (A-2) can be synthesized by adding diethylamine to an aqueous solution of potassium hydroxide and carbon disulfide to allow to react. Exemplified compound (A-12) can be synthesized by allowing carbon disulfide to react with piperidine.

According to the method of the present invention, the compound represented by formula (1) can be produced by allowing 1,4-benzoquinone or 1,2-benzoquinone to react with a dithiocarbamate compound represented by formula (2) in a polar solvent.

A preferable molar ratio of 1,4-benzoquinone or 1,2-benzoquinone to the compound represented by formula (2) is a range of 3/1 to 1/1, more preferably 2/1 to 1/1.

Reaction conditions for synthesizing the compound represented by formula (1) are explained in detail.

The reaction solvent that can be used in the synthesis is a polar solvent which may be protic or aprotic. Specific examples of the protic polar solvent include water, alcohols (e.g., methanol, ethanol, propanol, isopropyl alcohol, and butanol), carboxylic acids (e.g., acetic acid and propionic acid), glycol ethers (e.g., ethylene glycol monomethyl ether and ethylene glycol monoethyl ether), and the like. Among these, water, alcohols and carboxylic acids are preferable.

On the other hand, specific examples of the aprotic polar solvent include, amide-based solvents (e.g., N,N-dimethylformamide, N,N-dimethylacetamide, and N-methylpyrrolidone), acetates (e.g., methyl acetate and ethyl acetate), urea-based solvents (e.g., tetramethylurea and 1,3-dimethyl-2-imidazolidinone), ketones (e.g., acetone, and 2-butanone), ethers (e.g., diethyl ether, dioxane and tetrahydrofuran), nitriles (e.g., acetonitrile and propionitrile), dimethylsulfoxide, and the like. Among these, amide-based solvents, urea-based solvents and nitriles, each of which is relatively higher in polarity, are preferable. These solvents may be used alone or in combination of two or more kinds in consideration of solubility of the compound and so on. When two or more kinds of solvents are used in combination, it is preferable to use at least one kind of the protic polar solvent. Preferable examples of the combination include water/carboxylic acids/amide-based solvents, alcohols/carboxylic acids/amide-based solvents, water/carboxylic acids, water/amide-based solvents, alcohols/amide-based solvents, water/carboxylic acids/urea-based solvents, water/carboxylic acids/nitrites, water/carboxylic acids/dimethylsulfoxide, and the like. More preferable examples of the combination include water/carboxylic acids/amide-based solvents, alcohols/carboxylic acids/amide-based solvents, and the like.

Specific examples of the preferable combination of the reaction solvent include water/acetic acid/N-methylpyrrolidone, water/acetic acid/N,N-dimethylformamide, water/acetic acid/N,N-dimethylacetamide, water/acetic acid, water/N-methylpyrrolidone, methanol/acetic acid/N-methylpyrrolidone, ethanol/acetic acid/N,N-dimethylacetamide, water/acetic acid/acetonitrile, and the like. Specific examples of the more preferable combination include water/acetic acid/N-methylpyrrolidone, water/acetic acid/N,N-dimethylformamide, water/acetic acid/N,N-dimethylacetamide, water/acetic acid, water/N-methylpyrrolidone, and the like. These combinations may be properly selected in accordance with a substrate. When M of the compound represented by formula (2) is a hydrogen atom, it is necessary to use a base at least in an equivalent amount of the compound represented by formula (2).

The reaction temperature may be properly selected in accordance with a substrate. The reaction temperature is preferably from −10° C. to 80° C., more preferably from 0° C. to 60° C., and most preferably from 0° C. to 50° C. The reaction time may be properly selected in accordance with a substrate. The reaction time is preferably from 10 minutes to 5 hours, and more preferably from 20 minutes to 3 hours.

Further, the present invention relates to a method of producing a compound represented by formula (3). First, the compound represented by formula (3), which is the target compound of the present invention, is explained below.

In formula (3), R³, R⁴, R⁵ and R⁶ each independently represent a hydrogen atom or a monovalent substituent. At least one substituent of R³, R⁴, R⁵ and R⁶ represents a substituent having Hammett's substituent constant σₚ value of 0.2 or more.

The substituents of R⁵ and R⁶ as well as R³ and R⁴ are explained.

Examples of the monovalent substituent include halogen atoms (for example, fluorine atom, chlorine atom, bromine atom, and iodine atom), linear or branched alkyl groups having 1 to 20 carbon atoms (preferably 1 to 10 carbon atoms, for example, methyl and ethyl), aryl groups having 6 to 20 carbon atoms (preferably 6 to 10 carbon atoms, for example, phenyl and naphthyl), cyano group, carboxyl groups, alkoxycarbonyl groups having 1 to 20 carbon atoms (preferably 1 to 10 carbon atoms, for example, methoxycarbonyl), aryloxycarbonyl groups having 6 to 20 carbon atoms (preferably 6 to 10 carbon atoms, for example, phenoxycarbonyl), substituted or unsubstituted carbamoyl groups having 0 to 20 carbon atoms (preferably 0 to 10 carbon atoms, for example, carbamoyl, N-phenylcarbamoyl, and N,N-dimethylcarbamoyl), alkylcarbonyl groups having 1 to 20 carbon atoms (preferably 1 to 10 carbon atoms, for example, acetyl), arylcarbonyl groups having 6 to 20 carbon atoms (preferably 6 to 10 carbon atoms, for example, benzoyl), nitro group, substituted or unsubstituted amino groups having 0 to 20 carbon atoms (preferably 0 to 10 carbon atoms, for example, amino, dimethylamino, anilino), acylamino groups having 1 to 20 carbon atoms (preferably 1 to 10 carbon atoms, for example, acetamido and ethoxycarbonylamino), sulfonamido groups having 0 to 20 carbon atoms (preferably 0 to 10 carbon atoms, for example, methanesulfonamido), imido groups having 2 to 20 carbon atoms (preferably 2 to 10 carbon atoms, for example, succinimido and phthalimido), imino groups having 1 to 20 carbon atoms (preferably 1 to 10 carbon atoms, for example, benzylideneimino), hydroxy group, alkoxy groups having 1 to 20 carbon atoms (preferably 1 to 10 carbon atoms, for example, methoxy), aryloxy groups having 6 to 20 carbon atoms (preferably 6 to 10 carbon atoms, for example, phenoxy), acyloxy groups having 1 to 20 carbon atoms (preferably 1 to 10 carbon atoms, for example, acetoxy), alkylsulfonyloxy groups having 1 to 20 carbon atoms (preferably 1 to 10 carbon atoms, for example, methanesulfonyloxy), arylsulfonyloxy groups having 6 to 20 carbon atoms (preferably 6 to 10 carbon atoms, for example, benzenesulfonyloxy), sulfo groups, substituted or unsubstituted sulfamoyl groups having 0 to 20 carbon atoms (preferably 0 to 10 carbon atoms, for example, sulfamoyl and N-phenylsulfamoyl), alkylthio groups having 1 to 20 carbon atoms (preferably 1 to 10 carbon atoms, for example, methylthio), arylthio groups having 6 to 20 carbon atoms (preferably 6 to 10 carbon atoms, for example, phenylthio), alkylsulfonyl groups having 1 to 20 carbon atoms (preferably 1 to 10 carbon atoms, for example, methanesulfonyl), arylsulfonyl groups having 6 to 20 carbon atoms (preferably 6 to 10 carbon atoms, for example, benzenesulfonyl), and 4- to 7-membered heterocyclic groups (preferably 5- to 6-membered ring, for example, pyridyl and morpholino). The substituent may be further substituted. When plural substituents are present, they may be the same or different. Further, these substituents may be combined with each other to form a ring.

At least one substituent of R³, R⁴, R⁵ and R⁶ represents a substituent having Hammett's substituent constant σₚ value of 0.2 or more.

The expression “Hammett substituent constant σ value” used herein will be described. Hammett's rule is an empirical rule advocated by L. P. Hammett in 1935 for quantitatively considering the effect of substituents on the reaction or equilibrium of benzene derivatives and the appropriateness thereof is now widely recognized. The substituent constant determined in the Hammett's rule involves σₚ value and σₘ value. These values can be found in a multiplicity of general publications, and are detailed in, for example, “Lange's Handbook of Chemistry” 12th edition by J. A. Dean, 1979 (McGraw-Hill); “Kagaku no Ryoiki” special issue, No. 122, pp. 96 to 103, 1979 (Nankodo Publishing Co., Ltd.); and “Chemical Reviews”, 1991, Vol. 91, pp. 165-195.

The substituent having Hammett's substituent constant σₚ value of 0.2 or more in the present invention represents an electron-withdrawing group. Hammett's substituent constant σₚ value is preferably 0.25 or more, more preferably 0.3 or more, and particularly preferable 0.35 or more.

Examples of the group having Hammett's substituent constant σₚ value of 0.2 or more include a cyano group (0.66), a carboxyl group (—COOH:0.45), an alkoxycarbonyl group (—COOMe:0.45), an aryloxycarbonyl group (—COOPh:0.44), a carbamoyl group (—CONH₂:0.36), an alkylcarbonyl group (—COMe:0.50), an arylcarbonyl group (—COPh:0.43), an alkylsulfonyl group (—SO₂Me:0.72), and an arylsulfonyl group (—SO₂Ph:0.68). In the present specification, Me represents a methyl group and Ph represents a phenyl group. It is noted that the values in parentheses are σₚ values of representative substitutes extracted from Chem. Rev., Vol. 91 (1991), pp. 165-195.

R⁵ and R⁶ as well as R³ and R⁴ may bond together to form a ring. For example, when R³ and R⁴ bond together to form a ring, the σₚ values of R³ and R⁴ can not be determined. In the present invention, however, the σₚ value in the case of ring formation is determined, assuming that R³ and R⁴ are each substituted by the corresponding partial structure. For example, when 1,3-indandione ring is formed, it is assumed that R³ and R⁴ are each substituted by a benzoyl group. This determination is also applied to the case where R⁵ and R⁶ bond together to form a ring.

At least one substituent of R³, R⁴, R⁵ and R⁶ represents the substituent having Hammett's substituent constant σₚ value of 0.2 or more. It is preferable that any one of a combination of R³ and R⁴, and a combination of R⁵ and R⁶ is each the substituent having σₚ values of 0.2 or more. It is more preferable that any three of R³, R⁴, R⁵ and R⁶ is each the substituent having σₚ value of 0.2 or more. It is most preferable that each of R³, R⁴, R⁵ and R⁶ is the substituent having GP value of 0.2 or more.

At least one of R³, R⁴, R⁵ and R⁶ is preferably —CN, —COOR⁸, —CONR⁹R¹⁰, —COR¹¹ or —SO₂R¹² (Herein, R⁸, R⁹, R¹⁰, R¹¹ and R¹² each independently represent a hydrogen atom or a monovalent substituent.), more preferably —CN, —COOR⁸, —COR¹¹ or —SO₂R¹², further preferably —CN or —COOR⁸, and particularly preferable —CN.

It is especially preferable that at least one of R³, R⁴, R⁵ and R⁶ is an alkoxycarbonyl group in which the alkoxy moiety has one or more carbon atoms, more preferably from 1 to 20 carbon atoms, and further more preferably from 1 to 10. The alkoxy moiety may have a substituent at any position on the moiety. Examples of the substituent include the aforementioned substituents. As the alkoxy moiety of the alkoxycarbonyl group, there are exemplified a methoxy group, an ethoxy group, a propyloxy group, an isopropyloxy group, a butyloxy group, a hexyloxy group, a 2-ethylhexyloxy group, an octyloxy group, and a decyloxy group.

A combination of R⁵ and R⁶ as well as a combination of R³ and R⁴ may be any combination, as long as the combination satisfies the aforementioned limitation. However, it is more preferable that a combination of R³ and R⁴, and a combination of R⁵ and R⁶ are the same combination.

R⁵ and R⁶ as well as R³ and R⁴ may bond together to form a ring. The ring to be formed may be a saturated, or unsaturated, hydrocarbon ring, or hetero ring, with the proviso that neither dithiol ring nor dithioran ring is formed. Examples of the carbon-containing ring formed by bonding of R³ and R⁴ that is defined in formula (3) include a cyclopropane ring, a cyclobutane ring, a cyclopentane ring, a cyclohexane ring, a cycloheptane ring, a pyrrolidine ring, a tetrahydrofuran ring, a tetrahydrothiophene ring, an oxazoline ring, a thiazoline ring, a pyrroline ring, a pyrazolidine ring, a pyrazoline ring, an imidazolidine ring, an imidazoline ring, a piperidine ring, a piperadine ring, and a pyran ring. These rings may have a substituent at any position on the ring. Examples of the substituent include the aforementioned monovalent substituents. Further, examples of the substituent include divalent substituents such as a carbonyl group or an imino group. When plural substituents are present, they may be the same or different. Further, these substituents may bond together to form a ring, which results in a condensed ring or a spiro ring.

Specific preferred examples of the combination of R³ and R⁴, or R⁵ and R⁶ are shown in the following Tables 1-1 to 1-6, but the present invention is not limited thereto. In the present specification, Me represents a methyl group, Et represents an ethyl group, Bu represents a butyl group and Ph represents a phenyl group. The wavy line in Table indicates a binding site of the substituent on the hetero ring of formula (3).

TABLE 1
Specific examples of combinations

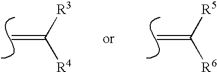

| | | | |
|---|---|---|---|
| 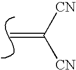 | 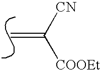 | 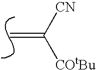 | 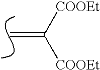 |

| | | | |
|---|---|---|---|
| 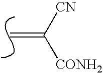 | 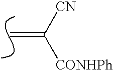 | 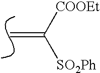 | 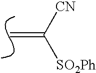 |

| | | | |
|---|---|---|---|
| 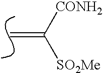 | 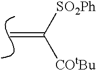 | 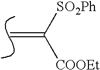 | 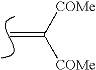 |

| | | | |
|---|---|---|---|
| 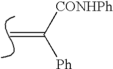 | 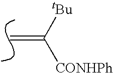 | 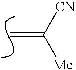 | 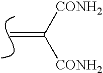 |

| | | |
|---|---|---|
| 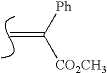 | 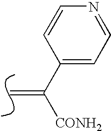 | 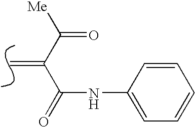 |

| | |
|---|---|
| 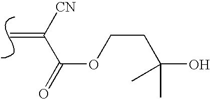 | 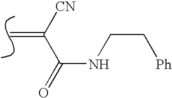 |

| 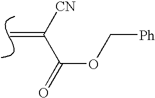 | 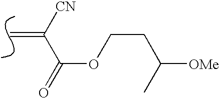 | |
|---|---|---|
| 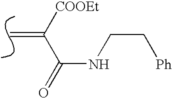 | 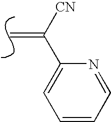 | 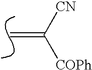 |

| | | |
|---|---|---|
| 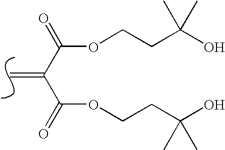 | 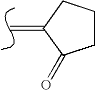 | 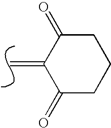 |

| | |
|---|---|
| 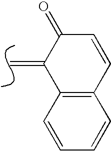 | 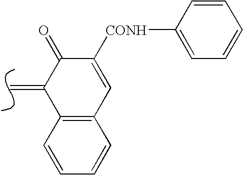 |

| | | |
|---|---|---|
| 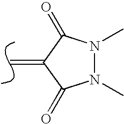 | 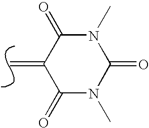 | 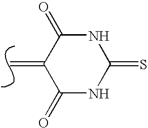 |

| | |
|---|---|
| 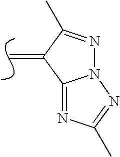 | 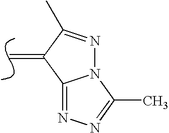 |

| | |
|---|---|
| 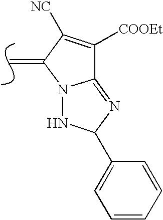 | 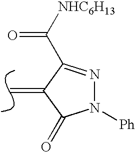 |

| | | |
|---|---|---|
| 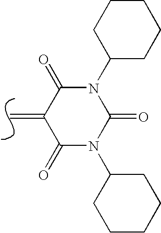 | 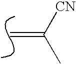 | 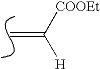 |

| | |
|---|---|
| 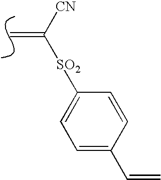 | 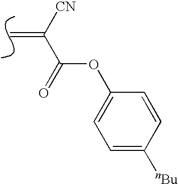 |

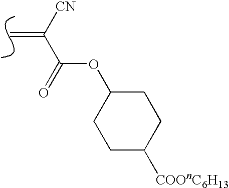

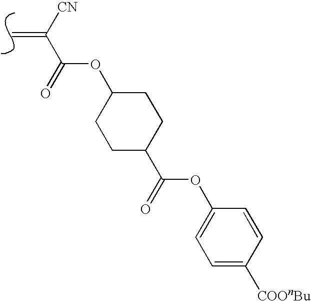

| | |
|---|---|
| 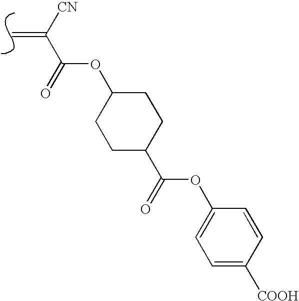 | 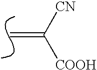 |

| | |
|---|---|
| 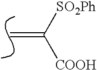 | 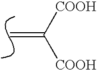 |

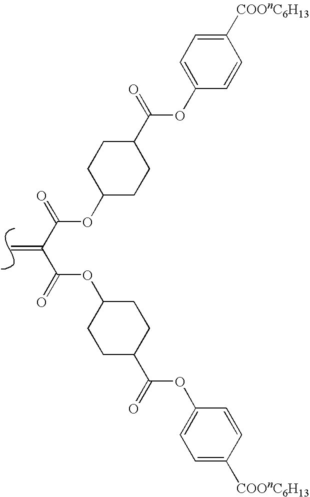

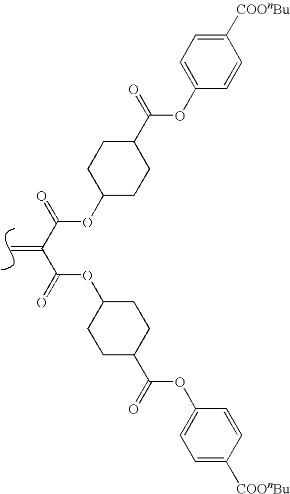

| | |
|---|---|
| 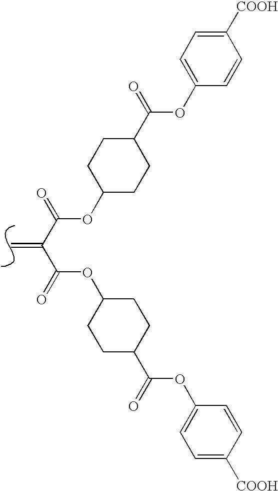 | 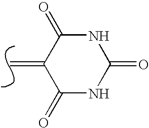 |

| | |
|---|---|
| 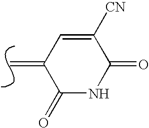 | 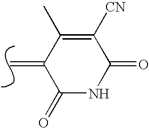 |

| | |
|---|---|
| 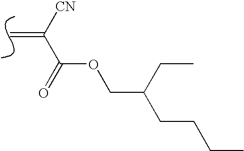 | 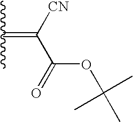 |

| | | |
|---|---|---|
| 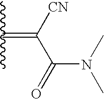 | 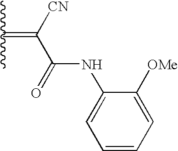 | 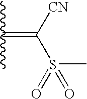 |

| | |
|---|---|
| 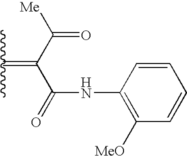 | 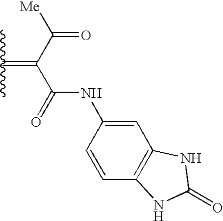 |

| | |
|---|---|
| 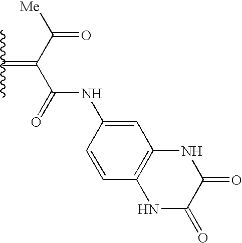 | 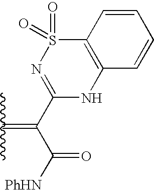 |

| | | |
|---|---|---|
| 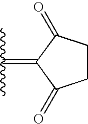 | 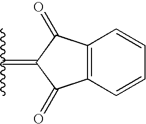 | 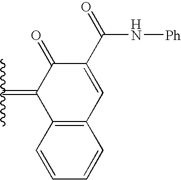 |

| | |
|---|---|
| 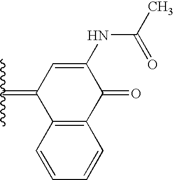 | 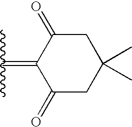 |

| | | |
|---|---|---|
| 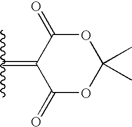 | 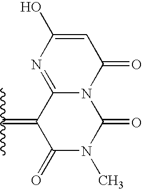 | 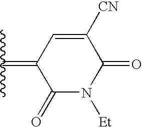 |

| | |
|---|---|
| 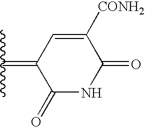 | 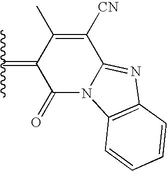 |

| | | |
|---|---|---|
| 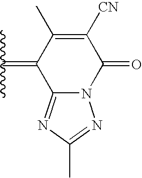 | 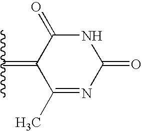 | 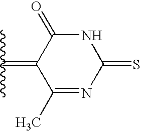 |

| | | |
|---|---|---|
| 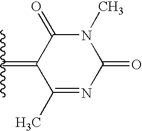 | 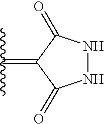 | 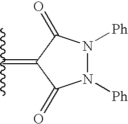 |

| | | |
|---|---|---|
| 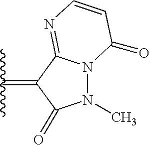 | 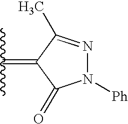 | 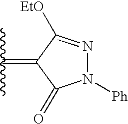 |

| | | |
|---|---|---|
| 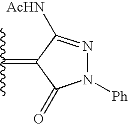 | 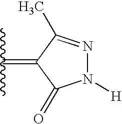 | 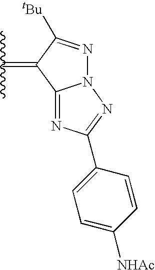 |

| | |
|---|---|
| 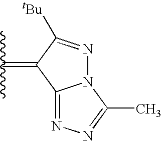 | 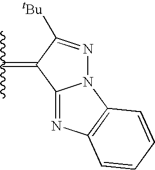 |

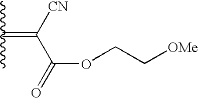

| | | |
|---|---|---|
| 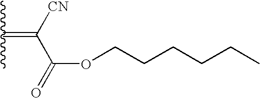 | 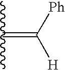 | 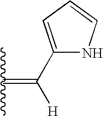 |

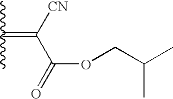

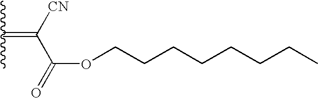

| | |
|---|---|
| 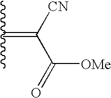 | 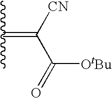 |

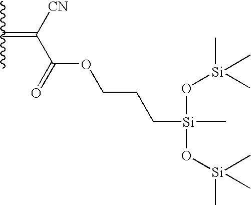

| | |
|---|---|
| 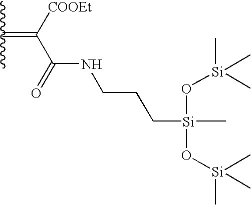 | 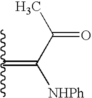 |

| | |
|---|---|
| 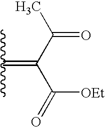 | 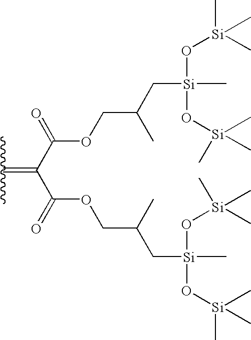 |

| | |
|---|---|
| 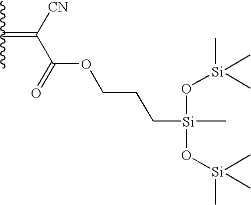 | 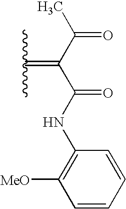 |

| | |
|---|---|
| 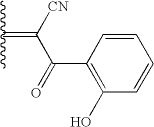 | 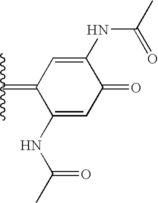 |

| | | |
|---|---|---|
| 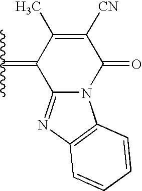 | 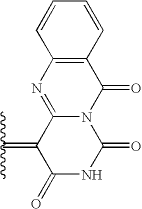 | 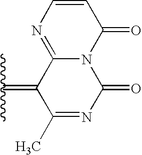 |

| | | |
|---|---|---|
| 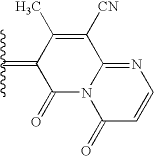 | 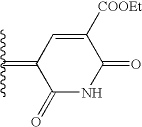 | 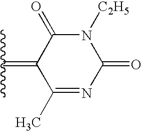 |

| | |
|---|---|
| 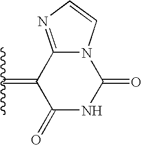 | 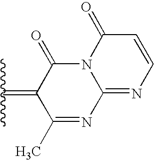 |

| | |
|---|---|
| 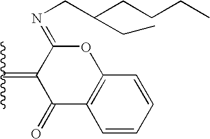 | 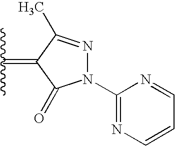 |

| | |
|---|---|
| 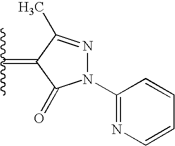 | 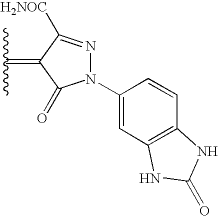 |

| | |
|---|---|
| 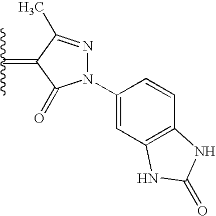 | 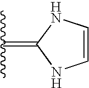 |

| | |
|---|---|
| 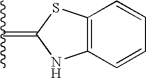 | 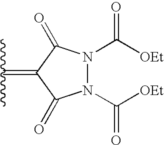 |

| | |
|---|---|
| 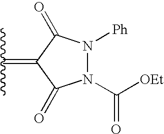 | 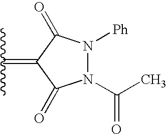 |

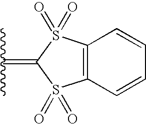

The compound represented by formula (3) includes compounds represented by formulae (3a) and (3b).

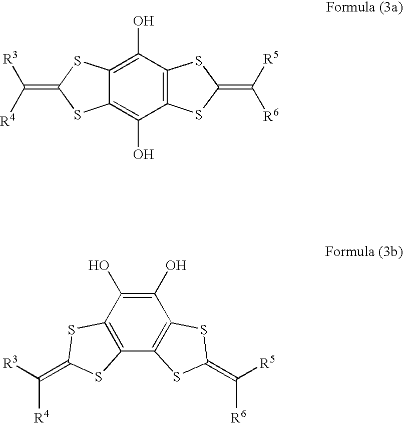

In formulae (3a) and (3b), R³, R⁴, R⁵ and R⁶ have the same meanings as those in formula (3), respectively, and the preferable ranges thereof are also the same.

Specific examples of the compound represented by formula (3) are shown below, but the present invention is not limited thereto.

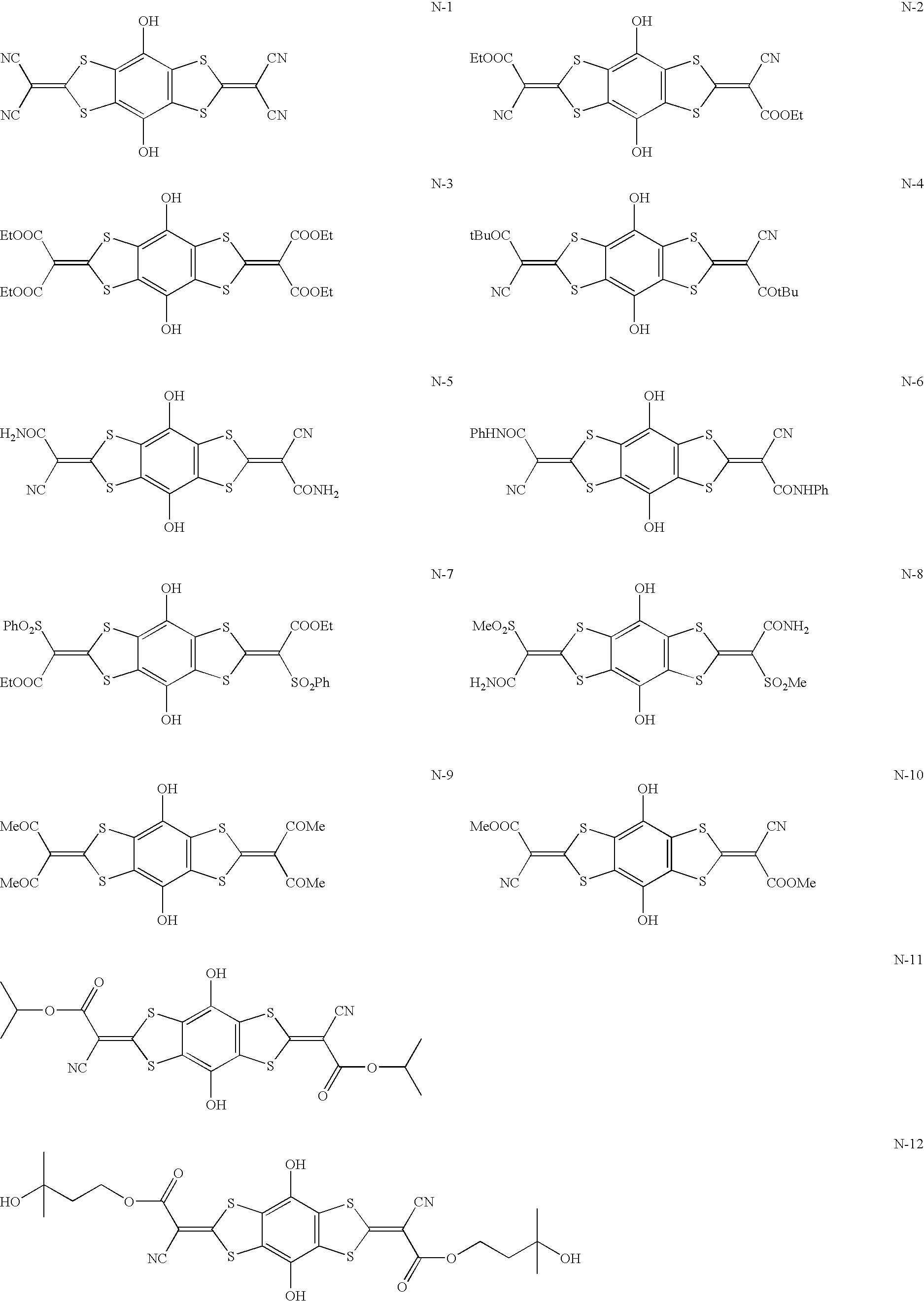

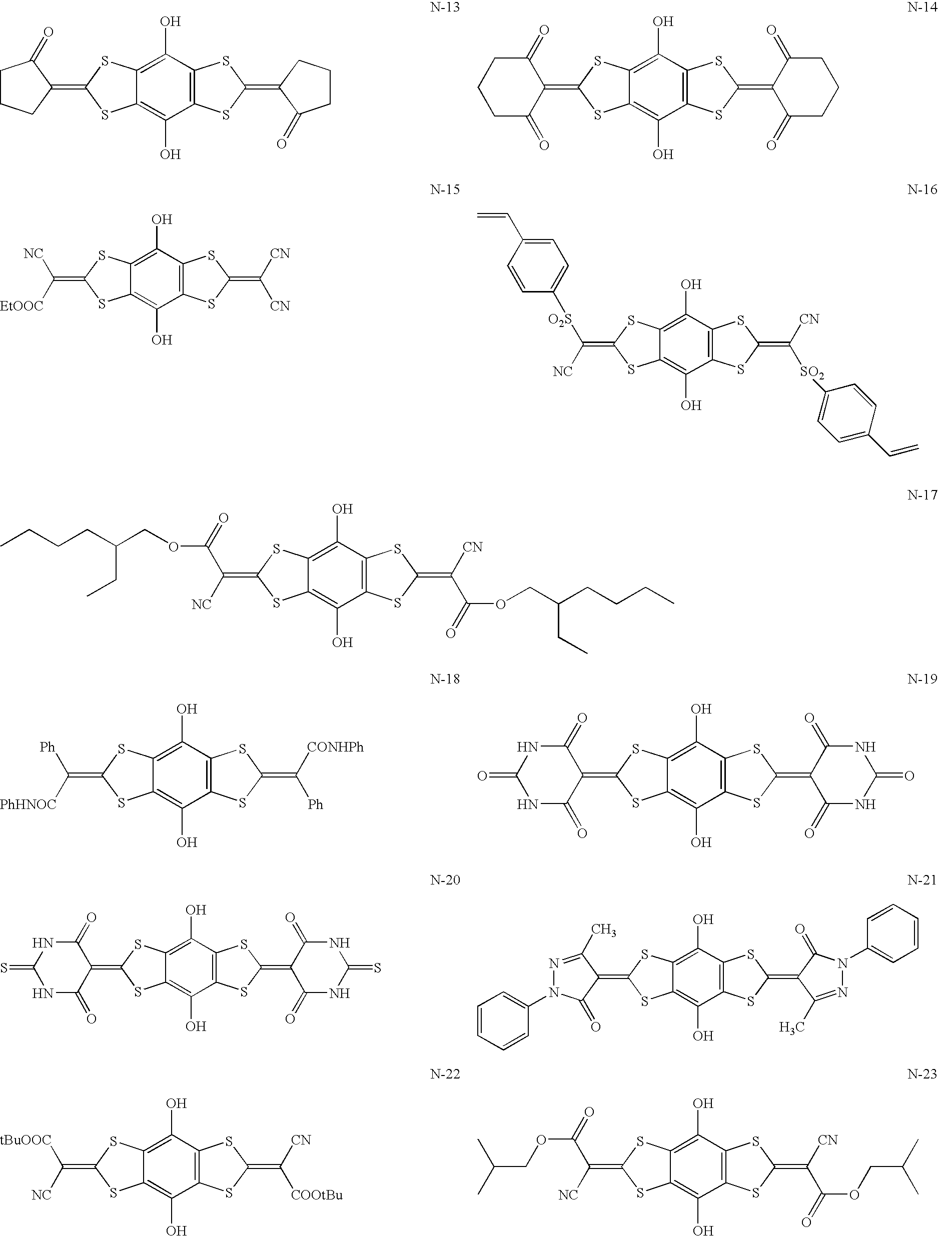

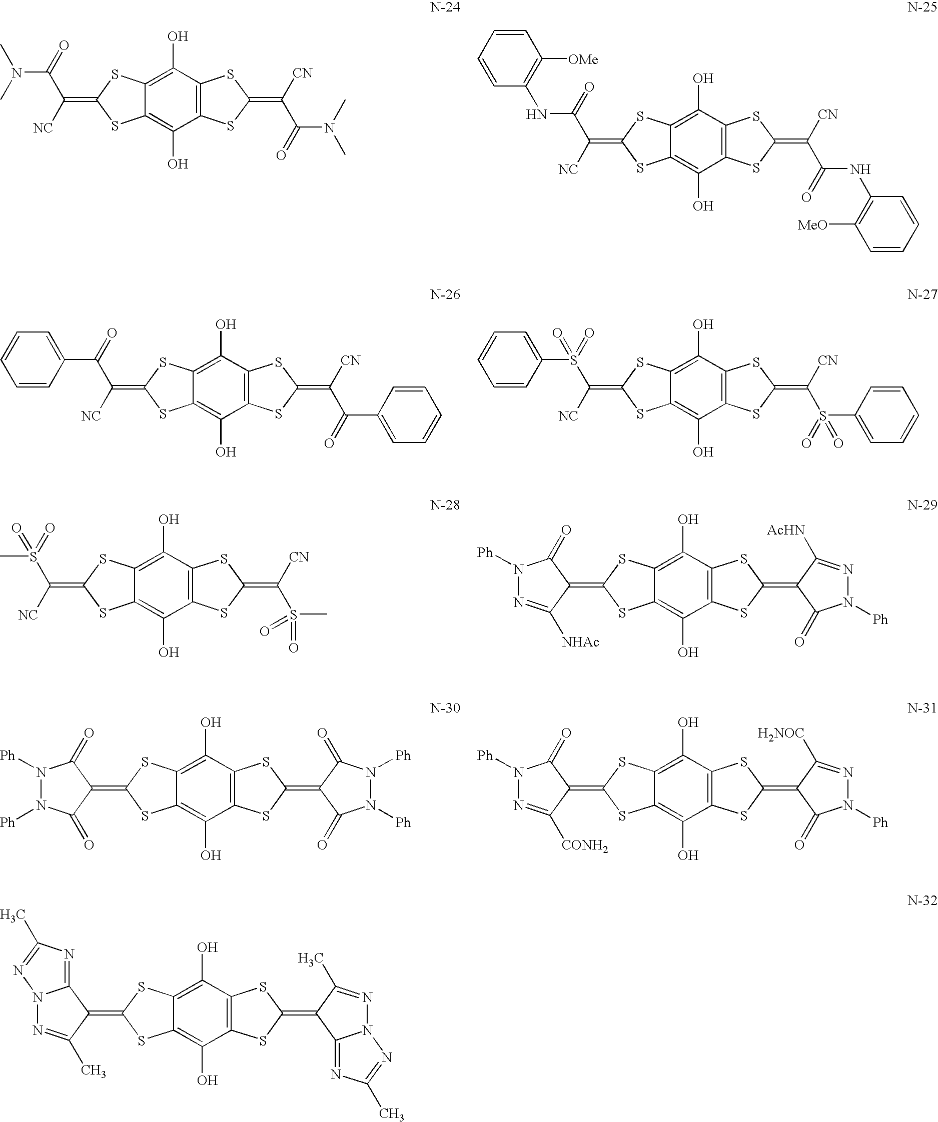

The compound represented by formula (3) may form a tautomer in accordance with the structure and the surroundings of the compound. Even though the compound is described by one of representative forms in the present specification, a tautomer that is different from the specification is also embraced in the compound represented by formula (3).

The compound represented by formula (3) may become a cation or an anion accompanying a proper counter ion in accordance with the structure and the surroundings of the compound. Even though the compound is described with a hydrogen ion as a counter cation, or a hydroxide ion as a counter anion in the present specification, the corresponding compound with a counter ion other than these ones is also embraced in the compound represented by formula (3). The counter ion may be a single ion, or a mixture of two or more ions having an arbitrary ratio.

The compound represented by formula (3) may have an isotopic element (such as ²H, ³H, ¹³C, ¹⁵N, ¹⁷O, or ¹⁸O).

Next, the compound represented by formula (4) or (5), which is a raw material for the compound represented by formula (3), is explained below.

In formulae (4) and (5), R³, R⁴, R⁵ and R⁶ have the same meanings as those in formula (3), respectively, and the preferable ranges thereof are also the same.

Specific examples of the compound represented by formula (4) or (5) are shown below, but the present invention is not limited thereto.

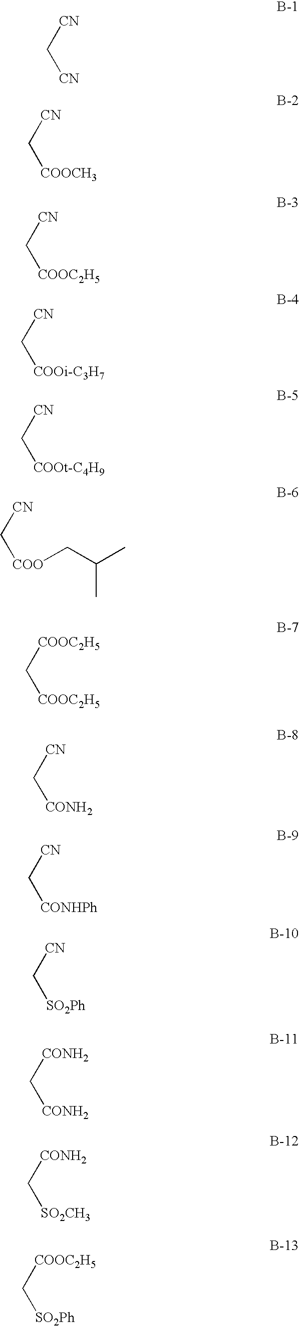

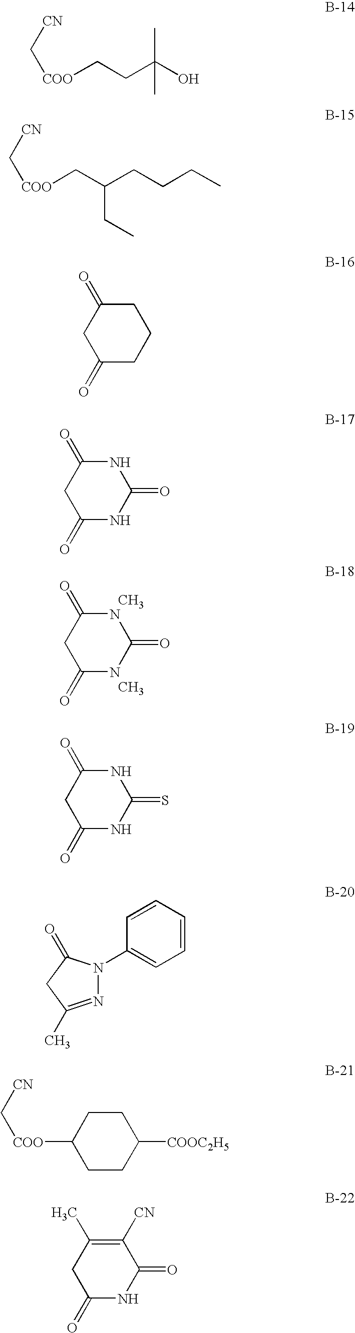

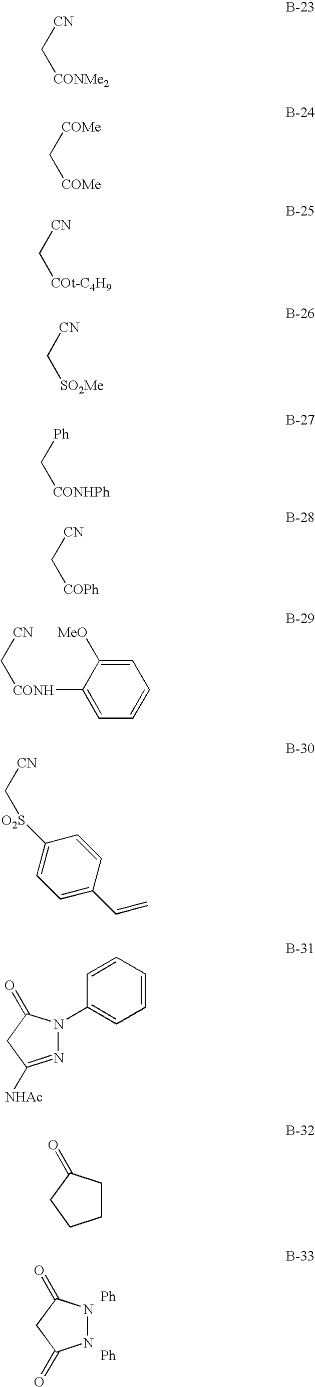

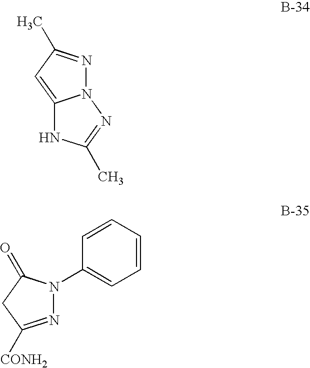

According to the method of the present invention, the compound represented by formula (3) is produced by allowing a compound represented by formula (1) to react with a compound represented by formula (4) in the first place, and allowing the resultant reaction mixture to react with a compound represented by formula (5).

A preferable molar ratio of the compound represented by formula (1) to the compound represented by formula (4) or (5) is a range of 1/2 to 1/4, more preferably 1/2 to 1/3.

Reaction conditions for synthesizing the compound represented by formula (3) are explained in detail.

Examples of the reaction solvent that can be used include water, alcohols (e.g., methanol, ethanol, isopropyl alcohol, and butanol), acetates (e.g., methyl acetate and ethyl acetate), amide-based solvents (e.g., N,N-dimethylformamide, N,N-dimethylacetamide, and N-methylpyrrolidone), ketones (e.g., acetone, and methyl ethyl ketone), ethers (e.g., diethyl ether, dioxane and tetrahydrofuran), glycol ethers (e.g., ethylene glycol monomethyl ether and ethylene glycol monoethyl ether), acetonitrile, acetic acid, dimethylsulfoxide, and the like. These solvents may be used alone or in combination of two or more kinds in consideration of solubility of the compound. Acids or bases can be added to the reaction solvent in accordance with a substrate.

The reaction temperature may be properly selected in accordance with a substrate. The reaction temperature is preferably from 20° C. to 150° C., more preferably from 30° C. to 120° C., most preferably from 40° C. to 100° C. The reaction time may be properly adjusted in accordance with a substrate. The reaction time is preferably from 30 minutes to 8 hours, and more preferably from 30 minutes to 6 hours.

According to the present invention, the compound represented by formula (3) can be produced by producing the compound represented by formula (1) by allowing a benzoquinone compound to react with a dichiocarbamate compound (the compound represented by formula (2)) followed by allowing the resultant reaction mixture to react with an active methylene compound (the compound represented by formula (4) or (5)). Namely, according to the present invention, the compound represented by formula (1) or (3) can be conveniently produced in a good yield by using no chloranil that is an environmentally harmful compound.

The compound represented by formula (1) or (3) is useful for organic electronic materials such as organic semi-conductors, synthetic intermediates for ultraviolet absorbents.

According to the method of the present invention, a bisbenzodithiol compound, which is useful for organic electronic materials, and synthetic intermediates such as ultraviolet absorbents, can be produced conveniently in a good yield using a safe and inexpensive raw material.

The present invention will be described in more detail based on the following examples, but the invention is not intended to be limited thereto.

### EXAMPLES

### Example 1

### Preparation of Exemplified Compound M-1

120 ml of N-methylpyrrolidone and 20 ml of water were added to 20 g (0.14 moles) of sodium dimethyl dithiocabamate dihydrate, and then 80 ml of acetic acid was added thereto under ice-cooling. 30.3 g (0.28 moles) of 1,4-benzoquinone was added by portions with stirring under ice-cooling. The stirring was continued at room temperature for 2 hours, and thereafter 200 ml of acetone was added to the reaction mixture. The deposited crystals were filtrated and washed with acetone. Thus, 25.7 g of Exemplified compound M-1 was obtained (Yield: 65.0%).

¹H NMR (CD₃COOD) δ(ppm) 6.70 (s, 8H), 3.68 (s, 12H)

### Example 2

### Preparation of Exemplified Compound M-2

100 ml of N-methylpyrrolidone was added to 40 g (0.113 moles) of potassium diethyl dithiocabamate (53% aqueous solution), and then 60 ml of acetic acid was added thereto with stirring under ice-cooling. 24.5 g (0.226 moles) of 1,4-benzoquinone was added bit by bit under ice-cooling. The stirring was continued at room temperature for 2 hours, and thereafter 150 ml of acetone was added to the reaction mixture. The deposited crystals were filtrated and washed with acetone. Thus, 23.9 g of Exemplified compound M-2 was obtained (Yield: 68.0%).

MS: m/z 402 (M⁺)

¹H NMR (CD₃COOD) δ(ppm) 6.70 (s, 8H), 3.99 (s, 8H), 1.51 (t, 12H)

### Example 3

### Preparation of Exemplified Compound M-2

### Alternative Method

38.4 g (0.17 moles) of sodium diethyl dithiocabamate trihydrate was dissolved in 19 ml of water and 180 ml of N-methylpyrrolidone, and then 90 ml of acetic acid was added thereto with stirring under ice-cooling. 18.4 g (0.17 moles) of 1,4-benzoquinone was added at an inner temperature of 25° C. or lower under ice-cooling over a period of 30 minutes. The stirring was continued at room temperature for 2 hours, and thereafter 9.2 g (0.085 moles) of 1,4-benzoquinone was added thereto. The stirring was continued at room temperature for additional 2 hours, and 60 ml of acetone was added to the reaction mixture. The deposited crystals were filtrated and washed with acetone. Thus, 35 g of Exemplified compound M-2 was obtained (Yield: 63%).

The obtained compound was identified as Exemplified compound M-2 by analyses of mass spectrometer and ¹H NMR.

### Comparative Example 1

### Preparation of Exemplified Compound M-15

A mixture solvent of ethanol/diethylether (2/1) was added to 24.6 g (0.1 moles) of chloranil. A solution in which 33.3 g (0.2 moles) of dimethylammonium dimethyldithiocabamate was dissolved in ethanol was added by drops into the mixture with stirring at room temperature. The mixture was heated to reflux for 1 hour, and thereafter the deposited crystals were filtrated. By recrystallization from a 0.5 N hydrochloric acid aqueous solution, 19.6 g of Exemplified compound M-15 was obtained (Yield: 47.0%).

### Example 4

### Preparation of Exemplified Compound N-4

100 ml of N-methylpyrrolidone was added to 12.4 g (0.02 moles) of Exemplified compound M-2 and 6.0 g (0.048 moles) of pivaloylacetonitrile. Then the mixture was stirred at an inner temperature of 80° C. for 4 hours under a nitrogen atmosphere. After cooling down to room temperature, 30 ml of 1N hydrochloric acid was added with stirring. The deposited crystals were filtrated and washed with water. Thus, 9.4 g of Exemplified compound N-4 was obtained (Yield: 98.0%).

¹H NMR (DMSO-d₆) δ(ppm) 1.32 (s, 18H)

### Example 5

### Preparation of Exemplified Compound N-12

50 ml of N-methylpyrrolidone was added to 8.0 g (0.0128 moles) of Exemplified compound M-2 and 4.8 g (0.028 moles) of 3-hydroxy-3-methylbutyl cyanoacetate. Then the mixture was stirred at an inner temperature of 80° C. for 3 hours under a nitrogen atmosphere. After cooling down to room temperature, 30 ml of ethyl acetate and 50 ml of water were added. 2.5 ml of concentrated hydrochloric acid was added with stirring. The deposited crystals were filtrated and washed with ethyl acetate and water. Thus, 7.3 g of Exemplified compound N-12 was obtained (Yield: 95.5%).

¹H NMR (DMSO-d₆) δ(ppm) 11.5-10.0 (br, 2H), 4.90-3.70 (br, 2H), 4.30 (t, 4H), 1.79 (t, 4H), 1.16 (s, 12H)

### Example 6

### Preparation of Exemplified Compound N-17

30 ml of N-methylpyrrolidone was added to 8.0 g (0.02 moles) of Exemplified compound M-2 and 5.7 g (0.029 moles) of 2-ethylhexyl cyanoacetate. Then the mixture was stirred at an inner temperature of 70° C. for 3 hours under a nitrogen atmosphere. After cooling down to room temperature, 40 ml of methanol followed by 8 ml of acetic acid were added thereto. The deposited crystals were filtrated and washed with methanol. Thus, 8.0 g of Exemplified compound N-17 was obtained (Yield: 96.0%).

¹H NMR (DMSO-d₆) δ(ppm) 4.25-4.05 (m, 4H), 1.70-1.54 (m, 2H), 1.45-1.20 (m, 16H), 0.97-0.78 (m, 12H)

### Example 7

### Preparation of Exemplified Compound N-19

1.24 g (0.002 moles) of Exemplified compound M-2 and 0.77 g (0.006 moles) of barbituric acid were suspended in 100 ml of dimethylsulfoxide, and then stirred for 5 hours on heating at 80° C. under a nitrogen gas stream. Thereafter the reaction mixture was cooled down to room temperature. A solid that was once dissolved, but thereafter deposited was filtrated and washed with dimethylsulfoxide and then with water, and then dried. Thus, 1.01 g of Exemplified compound N-19 (yellow crystal) was obtained (Yield: 99.0%).

Infrared absorption spectrum (cm⁻¹): 3430-3450 (s, br), 1718 (s), 1647 (s), 1431 (s), 1348 (m), 582 (m)

### Example 8

### Preparation of Exemplified Compound N-20

1.24 g (0.002 moles) of Exemplified compound M-2 and 0.90 g (0.006 moles) of thiobarbituric acid were suspended in 50 ml of dimethylsulfoxide, and then stirred for 4 hours on heating at 80° C. under a nitrogen gas stream. Thereafter the reaction mixture was cooled down to room temperature. A solid that was deposited was filtrated and washed with dimethylsulfoxide, water and methanol in this order, and then dried. Thus, 0.55 g of Exemplified compound N-20 (brownish yellow crystal) was obtained (Yield: 50.7%).

Infrared absorption spectrum (cm⁻¹): 3430-3450 (s, br), 3109 (m), 3018 (m), 2901 (m), 1660 (m), 1616 (s), 1531 (s), 1443 (s), 1161 (s)

### Example 9

### Preparation of Exemplified Compound N-21

1.24 g (0.002 moles) of Exemplified compound M-2 and 1.05 g (0.006 moles) of 3-methyl-1-phenyl-2-pyrazoline-5-one were suspended in 100 ml of dimethylsulfoxide, and then stirred on heating at 80° C. for 3 hours under a nitrogen gas stream. Thereafter the reaction mixture was cooled down to room temperature. To the obtained homogeneous solution, 10 ml of 1N hydrochloric acid aqueous solution was added. The deposited solid was filtrated and washed with water and then with methanol, and then dried. Thus, 1.90 g of Exemplified compound N-21 (yellow crystal) was obtained (Yield: 90.5%).

Infrared absorption spectrum (cm⁻¹): 3400-3420 (br, m), 1643 (m), 1594 (w), 1497 (s), 1335 (m)

### Example 10

### Preparation of Exemplified Compound N-1

Exemplified compound N-1 was prepared in the same manner as Example 4, except that acetonitrile was used instead of malononitrile.

Melting point: 397° C. or higher (decomposition)

MS: m/e 386 (M⁺)

Infrared absorption spectrum (cm⁻¹): 1460, 1450 (s), 2210, 1650 (br), 1360, 1310 (m), 3200, 2930, 1690, 1180, 1100,670, 500

### Example 11

### Preparation of Exemplified Compound N-2

Exemplified compound N-2 was prepared in the same manner as Example 6, except that ethyl cyanoacetate was used instead of 2-ethylhexyl cyanoacetate.

MS: m/e 480 (M⁺)

### Example 12

### Preparation of Exemplified Compound N-3

Exemplified compound N-3 was prepared in the same manner as Example 4, except that diethyl malonate was used instead of pivaloylacetonitrile.

MS: m/e 574 (M⁺)

### Example 13

### Preparation of Exemplified Compound N-7

Exemplified compound N-7 was prepared in the same manner as Example 5, except that ethyl phenysulfonylacetate was used instead of 3-hydroxy-3-methylbutyl cyanoacetate.

MS: m/e 710 (M⁺)

### Example 14

### Preparation of Exemplified Compound N-22

3.1 g of Exemplified compound M-2 was dispersed in 20 ml of N-methylpyrrolidone, and then 1.69 g of t-butyl cyanoacetate was added thereto. Thereafter, they were allowed to react at 80° C. for 6 hours. After cooling down to room temperature, 5 ml of acetic acid and 20 ml of methanol were added thereby to obtain a yellow powder. By recrystallization from methanol, Exemplified compound N-22 was obtained (Yield: 55%).

MS: m/e 536 (M⁺)

### Example 15

### Preparation of Exemplified Compound N-23

Exemplified compound N-23 was prepared in 49% yield in the same manner as the reaction using Exemplified compound M-2 in Example 14, except that iso-butyl cyanoacetate was used instead of t-butyl cyanoacetate.

MS: m/e 536 (M⁺)

### Example 16

### Preparation of Exemplified Compound N-24

Exemplified compound N-24 was prepared in 59% yield in the same manner as the reaction using Exemplified compound M-2 in Example 14, except that 2-cyano-N,N′-dimethylaceamide was used instead of t-butyl cyanoacetate.

MS: m/e 478 (M⁺)

### Example 17

### Preparation of Exemplified Compound N-25

Exemplified compound N-25 was prepared in 89% yield in the same manner as the reaction using Exemplified compound M-2 in Example 14, except that 2-cyano-N-(2-methoxyphenyl)acetamide was used instead of t-butyl cyanoacetate.

MS: m/e 634 (M⁺)

### Example 18

### Preparation of Exemplified Compound N-26

Exemplified compound N-26 was prepared in 85% yield in the same manner as the reaction using Exemplified compound M-2 in Example 14, except that benzoylacetonitrile was used instead of t-butyl cyanoacetate.

MS: m/e 544 (M⁺)

### Example 19

### Preparation of Exemplified Compound N-27

Exemplified compound N-27 was prepared in 44% yield in the same manner as the reaction using Exemplified compound M-2 in Example 14, except that phenylsulfonylacetonitrile was used instead of t-butyl cyanoacetate.

MS: m/e 616 (M⁺)

### Example 20

### Preparation of Exemplified Compound N-28

Exemplified compound N-28 was prepared in 53% yield in the same manner as the reaction using Exemplified compound M-2 in Example 14, except that methylsulfonylacetonitrile was used instead of t-butyl cyanoacetate.

MS: m/e 492 (M⁺)

### Example 21

### Preparation of Exemplified Compound N-29

3.1 g of Exemplified compound M-2 and 2.4 g of 3-acetylamido-1-phenyl-2-pyrazoline-5-one were suspended in 20 ml of dimethylsulfoxide, and then stirred for 5 hours on heating at 80° C. under a nitrogen gas stream. Thereafter the reaction mixture was cooled down to room temperature. To the obtained homogeneous solution, 10 ml of 1N hydrochloric acid aqueous solution was added. The deposited solid was filtrated and washed with water and then with methanol, and then dried. Thus, 3.0 g of Exemplified compound N-29 (yellow crystal) was obtained.

MS: m/e 688 (M⁺)

### Example 22

### Preparation of Exemplified Compound N-30

1.55 g of Exemplified compound M-2 and 1.40 g of 1,2-diphenyl-pyrazolidine-3,5-dione were suspended in 20 ml of dimethylsulfoxide, and then stirred for 3 hours on heating at 80° C. under a nitrogen gas stream. Thereafter the reaction mixture was cooled down to room temperature. To the obtained homogeneous solution, 5 ml of 1 N hydrochloric acid aqueous solution was added. The deposited solid was filtrated and washed with water and then with methanol, and then dried. Thus, 1.60 g of Exemplified compound N-30 (yellow crystal) was obtained.

MS: m/e 758 (M⁺)

### Example 23

### Preparation of Exemplified Compound N-31

2.48 g of Exemplified compound M-2 and 1.79 g of 3-carbamoyl-1-phenyl-2-pyrazolin-5-one were suspended in 20 ml of dimethylsulfoxide, and then stirred for 1.5 hours on heating at 80° C. under a nitrogen gas stream. Thereafter the reaction mixture was cooled down to room temperature. To the obtained homogeneous solution, 10 ml of 1 N hydrochloric acid aqueous solution was added. The deposited solid was filtrated and washed with water and then with methanol, and then dried. Thus, 4 g of Exemplified compound N-31 was obtained.

MS: m/e 660 (M⁺)

### Example 24

### Preparation of Exemplified Compound N-32

1.24 g of Exemplified compound M-2 and 0.82 g of Exemplified compound B-34 were suspended in 100 ml of dimethylsulfoxide, and then stirred for 12 hours on heating at 90° C. under a nitrogen gas stream. Thereafter the reaction mixture was cooled down to room temperature. To the obtained homogeneous solution, 10 ml of 1 N hydrochloric acid aqueous solution was added. The deposited solid was filtrated and washed with water and then with methanol, and then dried. Thus, 1 g of Exemplified compound N-32 was obtained.

MS: m/e 526 (M⁺)

### Comparative Example 2

### Preparation of Exemplified Compound N-1

To a solution in which 80 g (2 moles) of sodium hydroxide was dissolved in 800 ml of ethanol, a 100 ml of ethanol solution containing 66.1 g (1 mole) of malononitrile was added under ice-cooling, and then 76 g (1 mole) of carbon disulfide was added. After reaction of the resultant mixture at room temperature for 1 hour, the thus-obtained solid was filtrated and washed with ethanol. Thereby 166 g of di (sodium mercapto) methylenemalononitrile was obtained (Yield: 89.2%).

12.3 g (0.05 moles) of chloranil was dispersed in 100 ml of N,N-dimethylacetamide. To the resultant dispersion, a solution, in which 18.4 g (0.099 moles) of di (sodium mercapto) methylenemalononitrile was dissolved in 50 ml of water, was added under ice-cooling and allowed to react at room temperature for 5 hours. 50 ml of water was added to the reaction solution. The obtained solids were filtrated and washed with water. By recrystallization from THF-methanol, 10.1 g of Exemplified compound N-1 was obtained (Yield: 52%).

Melting point: 397° C. or higher (decomposition)

MS: m/e 386 (M⁺)

Infrared absorption spectrum (cm⁻¹): 1460, 1450 (s), 2210, 1650 (br), 1360, 1310 (m), 3200, 2930, 1690, 1180, 1100, 670, 500

According to the method of the present invention, a bisbenzodithiol compound that is useful for synthetic intermediates of organic electronic materials and ultraviolet absorbents can be conveniently produced in a good yield using a safe and inexpensive raw material.

Having described our invention as related to the present embodiments, it is our intention that the invention not be limited by any of the details of the description, unless otherwise specified, but rather be construed broadly within its spirit and scope as set out in the accompanying claims.

This non-provisional application claims priority under 35 U.S.C. § 119 (a) on Patent Application No. 2008-074640 filed in Japan on Mar. 21, 2008 and Patent Application No. 2008-187010 filed in Japan on Jul. 18, 2008, each of which are entirely herein incorporated by reference.

What we claim is:

1. A method of producing a compound represented by formula, (1) comprising: allowing 1,4-benzoquinone or 1,2-benzoquinone to react with a dithiocarbamate compound represented by formula (2) in a polar solvent: 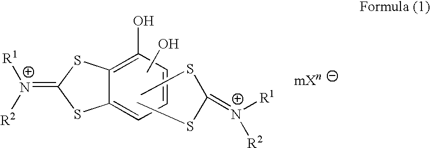
wherein R¹ and R² each independently represent a hydrogen atom, an alkyl group, an aryl group or a heterocyclic group; R¹ and R² may be the same or different and may be combined with each other to form a ring; X represents an ion necessary to neutralize the charge of the molecule; m represents an integer of 1 to 2; and n represents an integer of 1 to 2; 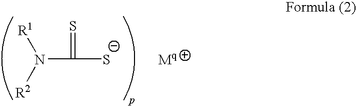
wherein R¹ and R² have the same meanings as those in formula (1); M represents a hydrogen atom, a metal atom or a conjugate acid of a base; p represents an integer of 1 to 4; and q represents an integer of 1 to 4.

2. The method of producing a compound represented by formula (1) according to claim 1, wherein X represents a monoanion of hydroquinone, a dianion of hydroquinone, a mono anion of catechol, a dianion of catechol, or a conjugate base of a protic acid.

3. The method of producing a compound represented by formula (1) according to claim 1, wherein the polar solvent is water, an alcohol, a carboxylic acid, an amide-based solvent, a urea-based solvent, a nitrile, or dimethylsulfoxide.

4. The method of producing a compound represented by formula (1) according to claim 1, wherein reaction of 1,4-benzoquinone or 1,2-benzoquinone with the dithiocarbamate compound represented by formula (2) is performed at a temperature in a range of 0° C. or higher and 60° C. or lower.

5. A method of producing a compound represented by formula (3), comprising: allowing a compound represented by formula (1) to react with a compound represented by formula (4), and allowing the resultant reaction mixture to react with a compound represented by formula (5): 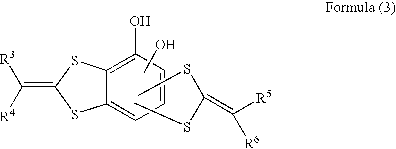
wherein R³, R⁴, R⁵ and R⁶ each independently represent a hydrogen atom or a monovalent substituent; and at least one substituent of R³, R⁴, R⁵ and R⁶ represents a substituent having Hammett's substituent constant σₚ value of 0.2 or more; 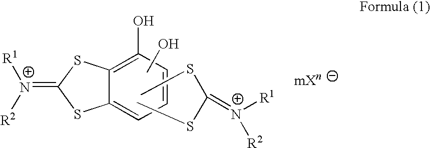
wherein R¹ and R² each independently represent a hydrogen atom, an alkyl group, an aryl group or a heterocyclic group; R¹ and R² may be the same or different and may be combined with each other to form a ring; X represents an ion necessary to neutralize the charge of the molecule; m represents an integer of 1 to 2; and n represents an integer of 1 to 2; 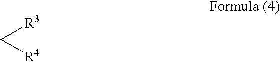
wherein R³ and R⁴ have the same meanings as those in formula (3); 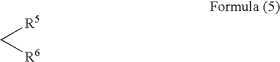
wherein R⁵ and R⁶ have the same meanings as those in formula (3).

6. The method of producing a compound represented by formula (3) according to claim 2, wherein X represents a monoanion of hydroquinone, a dianion of hydroquinone, a mono anion of catechol, a dianion of catechol, or a conjugate base of a protic acid.

7. The method of producing a compound represented by formula (3) according to claim 2, wherein the monovalent substituent represented independently by R³, R⁴, R⁵ and R⁶ is a cyano group, a carboxyl group, an alkoxycarbonyl group, an aryloxycarbonyl group, a carbamoyl group, an alkylcarbonyl group, an arylcarbonyl group, an alkylsulfonyl group, or an arylsulfonyl group.

8. The method of producing a compound represented by formula (3) according to claim 2, wherein reactions of the compound represented by formula (1) with the compound represented by formula (4), and the resultant reaction mixture with the compound represented by formula (5) are performed at a temperature in a range of 20° C. or higher and 150° C. or lower.
